(19) United States
(12) Patent Application Publication
(10) Pub. No.: US 2025/0302967 A1
(43) Pub. Date: Oct. 2, 2025

(54) PEPTIDE FORMULATIONS AND USES THEREOF

(71) Applicant: Amytrx Therapeutics, Inc., Birmingham, AL (US)

(72) Inventors: Sylvia K. Gonda, Birmingham, AL (US); Vernon L. Alvarez, Birmingham, AL (US); Matthew A. Gonda, Birmingham, AL (US)

(21) Appl. No.: 18/865,280

(22) PCT Filed: May 12, 2023
(86) PCT No.: PCT/US2023/066931

§ 371 Date: Nov. 12, 2024

Related U.S. Application Data
(60) Provisional application No. 63/341,398, filed on May 12, 2022.
(60) Provisional application No. 63/400,351, filed on Aug. 23, 2022.

(51) Int. Cl. A61K 47/42; A61K 9/06; A61K 47/10; A61K 47/14; A61K 47/18; A61K 47/32; A61K 47/38; A61P 17/00

(52) U.S. Cl. CPC A61K 47/42; A61K 9/06; A61K 47/10; A61K 47/14; A61K 47/18; A61K 47/32; A61K 47/38; A61P 17/00

(57) ABSTRACT

Active pharmaceutical agent-containing formulations, for example, peptide-containing formulations, and uses thereof are provided herein, including uses in treating leukocyte associated diseases.

### RELATED APPLICATIONS

This application claims priority of U.S. Provisional Patent Application Nos. 63/400,351, filed Aug. 23, 2022, and 63/341,398, filed May 12, 2022, the entire content of each of which is incorporated herein by reference.

### SEQUENCE LISTING

A Sequence Listing is submitted herewith and incorporated by reference herein as an XML file created on May 8, 2023, entitled “1959919-00008_Sequence_Listing.xml” and having a size of 78 KB.

### SUMMARY

Topically applied skin formulations have been discovered that are useful drug delivery vehicles. It has been discovered that topical application of formulations provided herein results in delivery of a compound, an active agent of the formulation, comprising a particular amino acid sequence, to leukocytes. The formulations are useful in treating leukocyte associated diseases, immune, inflammatory, or neurodegenerative diseases. Independent of the active agent, the formulations provided herein are useful in delivering one or more active agents to a subject in need thereof and treating a disease in the subject that the one or more active agents are useful in treating.

Thus, topical skin formulations and uses thereof are provided herein, including peptide-containing formulations and their uses in treating leukocyte associated diseases.

### DETAILED DESCRIPTION

### Definitions

Certain terms, whether used alone or as part of a phrase or another term, are defined below.

The articles “a” and “an” refer to one or to more than one of the grammatical object of the article.

Numerical values relating to measurements are subject to measurement errors that place limits on their accuracy. For this reason, all numerical values provided herein, unless otherwise indicated, are to be understood as being modified by the term “about.” Accordingly, the last decimal place of a numerical value provided herein indicates its degree of accuracy. Where no other error margins are given, the maximum margin is ascertained by applying the rounding-off convention to the last decimal place or last significant digit when a decimal is not present in the given numerical value.

The term “amelioration” means a lessening of severity of at least one indicator of a condition or disease, such as a delay or slowing in the progression of one or more indicators of a condition or disease. The severity of indicators may be determined by subjective or objective measures which are known to those skilled in the art.

The terms “formulation” and “pharmaceutical formulation” refer to a mixture of at least one compound described herein with a pharmaceutically acceptable carrier. The pharmaceutical formulation facilitates administration of the compound to a patient or subject.

The terms “effective amount” and “therapeutically effective amount” refer to an amount of active ingredient, such as a compound described herein, administered to a subject, either as a single dose or as part of a series of doses, which produces a desired effect. In general, the effective amount can be estimated initially either in cell culture assays or in mammalian animal models, for example, in non-human primates, murines (for example, mice), leporines (for example, rabbits), canines (for example, dogs), bovines, assinines, equines, cervines or elaphines or rusines, felines, ursines, ovines (for example, sheep), hircines (for example, goats), or pigs. The animal model may also be used to determine the appropriate concentration range and route of administration. Such information can then be used to determine useful doses and routes for administration in non-human subjects and human subjects.

The term “pharmaceutically acceptable carrier” means a pharmaceutically acceptable material, composition or carrier, such as a liquid filler, solid filler, stabilizer, dispersing agent, suspending agent, diluent, excipient, thickening agent, solvent, or encapsulating material, involved in carrying or transporting at least one compound described herein within or to the patient such that the compound may perform its intended function. A given carrier must be “acceptable” in the sense of being compatible with the other ingredients of a particular formulation, including the compounds described herein, and not injurious to the patient. Other ingredients that may be included in the pharmaceutical formulations described herein are known in the art and described, for example, in “Remington's Pharmaceutical Sciences” (Genaro (Ed.), Mack Publishing Co., 1985), the entire content of which is incorporated herein by reference.

The term “pharmaceutically acceptable salt” refers to derivatives of the disclosed compounds wherein the parent compound is modified by converting an existing acid or base moiety to its salt form. Pharmaceutically acceptable salts can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two solvents. Lists of suitable salts are found in “Handbook of Pharmaceutical Salts: Properties, Selection, and Use” (P. Henrich Stahl & Camille G. Wermuth (Eds.), VHCA & Wiley-VCH, 2002), the entire content of which is incorporated herein by reference.

The term “plasma” as used herein refers to whole blood depleted of red blood cells.

The term “refractory disease” refers to a disease that continues to progress during treatment with a pharmaceutical ingredient other than the compounds provided herein, partially responds to the other treatment, or transiently responds to the other treatment. The term may be applied to each of the diseases referred to herein.

The terms “treatment” or “treating” refer to the application of one or more specific procedures used for the amelioration of a disease. A “prophylactic” treatment, refers to reducing the rate of progression of the disease or condition being treated, delaying the onset of that disease or condition, or reducing the severity of its onset.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided herein is intended merely to better illuminate the described subject matter and does not pose a limitation on the scope of the subject matter otherwise claimed. No language in the specification should be construed as indicating any non-claimed element essential to practicing the described subject matter.

Groupings of alternative elements or embodiments of this disclosure are not to be construed as limitations. Each group member may be referred to and claimed individually or in any combination with other members of the group or other elements found herein. Furthermore, a recited member of a group may be included in, or excluded from, another recited group for reasons of convenience or patentability.

Reference made to a patent document or other publication in this specification serves as an incorporation herein by reference of the entire content of such document or publication.

Embodiments of this disclosure are illustrative. Accordingly, the present disclosure is not limited to that precisely as shown and described.

### Formulations

Topically applied skin formulations have been discovered that are useful drug delivery vehicles. It has been discovered that topical application of formulations provided herein results in delivery of a compound, an active agent of the formulation, comprising a particular amino acid sequence, to leukocytes. The formulations are useful in treating leukocyte associated diseases. In some embodiments, the compound comprises a sequence derived from a fibroblast growth factor (FGF) signal sequence, for example, an FGF4 signal sequence comprising SEQ ID NO: 1 (AVALLPAVLLALLA).

It has also been discovered that the compounds provided herein, or other active pharmaceutical agents, may be prepared in a formulation suitable for topical application, wherein the formulations comprise about 0.5-1% carbomer, about 85% or more of water, optionally including about 0.05-0.3 paraben, and are stable (for example, their viscosity remains essentially unchanged from that as formulated) at room temperature. In some embodiments, topical formulations provided herein may be used alone or as a component of a topical patch, for example a controlled release topical patch.

In some embodiments, provided herein are formulations, comprising at least one active pharmaceutical agent, for example a compound comprising an amino acid sequence of a formula selected from:

(SEQ ID NO: 2)
AAVAPAVX¹⁶X¹⁶AX¹⁶X¹⁶A;
(SEQ ID NO: 3)
AAVALLPAVLLALLA;
(SEQ ID NO: 4)
X¹X¹AAVALLPAVLLALLAX¹X¹;
(SEQ ID NO: 5)
KKAAVALLPAVLLALLAKK;
(SEQ ID NO: 6)
RRAAVALLPAVLLALLARR;
(SEQ ID NO: 7)
RRAAVALLPAVLLALLARK;
(SEQ ID NO: 8)
RKAAVALLPAVLLALLARKY;
(SEQ ID NO: 9)
AAVALLPAVLLALLAPCVQRKRQKLMPC;
(SEQ ID NO: 10)
X¹X²AAX³AX⁴X⁵X¹⁷AX⁶X⁷X⁸AX⁹X¹⁰A(P)ₙX¹¹X¹²(X¹³)ₙ;
(SEQ ID NO: 11)
X¹X²AAVALLPAVLLALLAPX¹¹X¹²(X¹³)ₙ;
(SEQ ID NO: 12)
X¹X²AAX³AX⁴X⁵X¹⁷AX⁶X⁷X⁸AX⁹X¹⁰APX¹¹X¹²(X¹³),
CVQRKRQKLMPC;
(SEQ ID NO: 13)
AAVALLPAVLLALLAPVQRKRQKLMP;
(SEQ ID NO: 14)
AAVALLPAVLLALLAPCVQRKRQKLMPC
(cyclized by SS bond at C¹⁷ and C²⁸)
(SEQ ID NO: 15)
AAVAPAVX¹⁶X¹⁶AX¹⁶X¹⁶AP;
(SEQ ID NO: 16)
AAVALLPAVLLALLAP;
(SEQ ID NO: 17)
(X¹⁴)ₙ(X¹⁵)ₙAAVALLP(X¹⁴)ₘ;
(SEQ ID NO: 18)
(X¹⁴)ₙ(X¹⁵)ₙAVLLALLAP(X¹⁴)ₘ;
(SEQ ID NO: 20)
(X¹⁴)ₙ(X¹⁵)ₙAAVALLAAVLLALLAP(X¹⁴)ₘ;
(SEQ ID NO: 21)
TTGTLLPGVLLALVVA;
(SEQ ID NO: 22)
(X¹⁴)ₙ(X¹⁵)ₙTTGTLLPGVLLALVVA(X¹⁴)ₘ;
(SEQ ID NO: 23)
TTGTLLPRVLLALVVA;
(SEQ ID NO: 24)
(X¹⁴)ₙ(X¹⁵)ₙTTGTLLPGVLLALVVA(X¹⁴)ₘ;
(SEQ ID NO: 25)
(X¹⁴)ₙ(X¹⁵),TTGTLLP(X¹⁴)ₘ;
(SEQ ID NO: 26)
(X¹⁴)ₙ(X¹⁵)ₙVLLALVVA(X¹⁴)ₘ;
(SEQ ID NO: 28)
RAAVALLPAVLLALLAPY(X¹⁴)ₘ;
(SEQ ID NO: 29)
RAAVALLPAVLLAVLAPY(X¹⁴)ₘ;
(SEQ ID NO: 30)
(X¹⁴)ₙ(X¹⁵)ₙAAVALLPAVLLALLAPDVRKRQDLEQ KM(X¹⁴)ₙ(Y)ₘ;
(SEQ ID NO: 31)
(X¹⁴)ₙ(X¹⁵)ₙAAVALLPAVLLALLAP(X¹⁴)ₘ;
(SEQ ID NO: 32)
(X¹⁴)ₙ(X¹⁵)ₙAAVALLAAVLLALLAP(X¹⁴)ₘ;
(SEQ ID NO: 33)
(X¹⁴)ₙ(X¹⁵)ₙTTGTLLPGVLLALVVA(X¹⁴)ₘ;
(SEQ ID NO: 34)
(X¹⁴)ₙ(X¹⁵)ₙTTGTLLPRVLLALVVA(X¹⁴)ₘ;
(SEQ ID NO: 35)
(X¹⁴)ₙ(X¹⁵)ₙTTGTLLP(X¹⁴)ₘ;
(SEQ ID NO: 36)
(X¹⁴)ₙ(X¹⁵)ₙGVLLALVVA(X¹⁴)ₘ;
(SEQ ID NO: 37)
(X¹⁴)ₙ(X¹⁵)ₙAAVALLPAVLLALLAPCVQKRQKLMPC;
(SEQ ID NO: 38)
KKAAVALLPAVLLALLAPKK;
(SEQ ID NO: 39)
RRAAVALLPAVLLALLAPRR;
(SEQ ID NO: 40)
RRAAVALLPAVLLALLAPRK;
(SEQ ID NO: 41)
RKAAVALLPAVLLALLAPRKY;
(SEQ ID NO: 42)
KRAAVALLPKK;
(SEQ ID NO: 43)
KRAVLLALLAPKK;
(SEQ ID NO: 44)
KRAAVALLAAVLLALLAPKK;
(SEQ ID NO: 45)
KRTTGTLLPGVLLALVVAKK;
(SEQ ID NO: 46)
KRTTGTLLPGVLLALVVAKK;
(SEQ ID NO: 47)
KRTTGTLLPKK;
(SEQ ID NO: 48)
KRVLLALVVAKK;
(SEQ ID NO: 49)
KRAAVALLPKK;
(SEQ ID NO: 50)
AAVALLPAVLLALLAP;
(SEQ ID NO: 51)
AAVALLPAVLLALLAPCYVQRKRQKLMPC;
(SEQ ID NO: 52)
AAVALLPAVLLAVLAPCVQRKRQKLMPC;
(SEQ ID NO: 53)
AAVALLPAVLLALLAPCVQRDEQKLMPC;
or
(SEQ ID NO: 54)
AAVALLPAVLLAVLAPCVQRDEQKLMPC;

or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
X¹⁴ is lysine;
X¹⁵ is arginine;
X¹⁶ is leucine, isoleucine, or norleucine;
X¹⁷ is proline or alanine;
n is 0 or 1; and
m is 0 or 2.

In some embodiments, provided herein are formulations, comprising a compound comprising an amino acid sequence of the formula:

(SEQ ID NO: 55)
X¹X²AAX³AX⁴X⁵PAX⁶X⁷X⁸AX⁹X¹⁰A(P)ₙX¹¹X¹²(X¹³)ₘ

or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
m is 0, 1, or 2; and
n is 0 or 1.

In some embodiments of the formulations provided herein, the formulation is a formulation for topical administration. In some embodiments, the formulation is topically applied to the skin of a subject. In some embodiments, the active pharmaceutical agent (for example, a compound provided herein) is about 0.05 to about 2.0% w/w of the formulation. In some embodiments, the compound is about ≥70% pure, about ≥80% pure, ≥90% pure, ≥95% pure, or ≥98% pure. In some embodiments, the formulation comprises about 0.11% w/w, 0.33% w/w, or 1.1% w/w of the compound. In some embodiments, the compound or formulation, or both, is stable for up to one, two, three, or four months, or more, at about 20-25° C. In some embodiments, the compound or formulation, or both, is stable for at least 6 weeks, or up to about one year at about 30° C. In some embodiments, to formulation is an ointment formulation, a cream formulation, a cold cream formulation, a gel formulation, a paste formulation, a liposomal formulation, a microemulsion formulation, or a nanoparticle formulation. In some embodiments, the formulation is a cream formulation. In some embodiments, the formulation further comprises one or more of propylene glycol, methylparaben, propylparaben, isopropyl palmitate, polyethylene glycol, polyvinyl carboxy polymer, methylcellulose, polyoxyethylene (20) sorbitan monooleate, triethanolamine, or water. In some embodiments, the water is present in the formulation in at least about 87% w/w. In some embodiments, the formulation is housed in a container that protects the formulation from exposure to direct light or from exposure to heat. In some embodiments, the formulation comprises about 0.11% w/w, 0.33% w/w, or 1.1% w/w of the compound. In some embodiments, the amount of compound present is with respect to the mass of the free base of the compound. In some embodiments, the amount of compound present is with respect to a salt form of the compound.

In some embodiments, the formulation comprises: about 0.05-1.5% w/w of leukocyte-targeting compound; about 0.05-0.2% w/w methyl paraben; about 0.01-0.05% w/w propyl paraben; about 1.5-2.5% w/w propylene glycol; about 1.5-2.5% w/w isopropyl palmitate; about 0.4-0.6% w/w PEG 8000; about 0.4-0.6% w/w PEG 400; about 0.5-1.0% w/w carbomer 974P; about 0.5-0.7% w/w methyl cellulose 4000 cP; about 0.4-0.6% w/w polysorbate 80; about 0.7-0.9% w/w triethanolamine; and at least 87% w/w water (for example, about 91.0% w/w, about 91 to about 92% w/w, or about 88 to about 94% w/w water). In some embodiments, the formulation comprises: about 0.16% w/w methyl paraben; about 0.04% w/w propyl paraben; about 2% w/w propylene glycol; about 2% w/w isopropyl palmitate; about 0.5% w/w PEG 8000; about 0.5% w/w PEG 400; about 0.8% w/w carbomer 974P; about 0.6% w/w methyl cellulose 4000 cP; about 0.5% w/w polysorbate 80; about 0.8% w/w triethanolamine; and at least 87% w/w water (for example, about 91.0% w/w, about 91 to about 92% w/w, or about 88 to about 94% w/w water). In some embodiments, the formulation comprises about 87+0.5, about 88+0.5, about 89+0.5, about 90+0.5, about 91+0.5, about 92+0.5, about 93+0.5, or about 94+0.5 w/w or more of water.

In some embodiments regarding the compound of the formulation, X³ and X⁶ are valine. In some embodiments, X⁴, X⁵, X⁷, X⁸, X⁹, and X¹⁰ are each, independently, leucine, isoleucine, or norleucine. In some embodiments, the compound comprises an amino acid sequence of the formula: X¹X²AAVALLPAVLLALLA(P)ₘX¹¹X¹²(X¹³)ₙ, or a pharmaceutically acceptable salt thereof. In some embodiments, the compound comprises at least one ornithine, isoleucine, or norleucine. In some embodiments, the compound comprises an amino acid sequence selected from KKAAVALLPAVLLALLAKK, RRAAVALLPAVLLALLARR, RRAAVALLPAVLLALLARK, RKAAVALLPAVLLALLARKY, KKAAVALLPAVLLALLAPKK, RRAAVALLPAVLLALLAPRR, RRAAVALLPAVLLALLAPRK, or RKAAVALLPAVLLALLAPRKY. In some embodiments, the compound further comprises the amino acid sequence CVQRKRQKLMPC, for example at the carboxy terminus. In some embodiments, the compound comprises an amino acid sequence of the formula: X¹X²AAX³AX⁴X⁵PAX⁶X⁷X⁸AX⁹X¹⁰A(P)ₘX¹¹X¹²(X¹³)ₙCVQRKRQKLMPC, or a pharmaceutically acceptable salt thereof. In some embodiments, the compound comprises at least one iodine. In some embodiments, the at least one iodine is selected, independently, from ¹²³I, ¹²⁴I, ¹²⁵I, or ¹³¹I. In some embodiments, the compound comprises at least one D-amino acid. In some embodiments, the compound comprises an amino acid sequence AAVALLPAVLLALLACVQRKROKLMPC. In some embodiments, the compound comprises an amino acid sequence AAVALLPAVLLALLAPCVQRKROKLMPC. In some embodiments, the compound comprises a cyclic peptide. In some embodiments, the compound comprises a cysteine at position 16 and 17 of the amino acid sequence, or at position 17 and 28 of the amino acid sequence, and C¹⁶ and C²⁷ or C¹⁷ and C²⁸ are covalently linked. In some embodiments, the compound comprises two cysteine amino acids that are covalently linked by a disulfide bond. In some embodiments, the compound is covalently linked to an active pharmaceutical agent, which may be referred to herein as a conjugate. In some embodiments, the active pharmaceutical agent linked to the compound is selected from a peptide, a protein (for example, an antibody), a small molecule therapeutic, an oligomer (for example, an oligonucleotide, for example, an RNA or a DNA), or a radionucleotide, or a combination thereof.

In some embodiments, the compound is

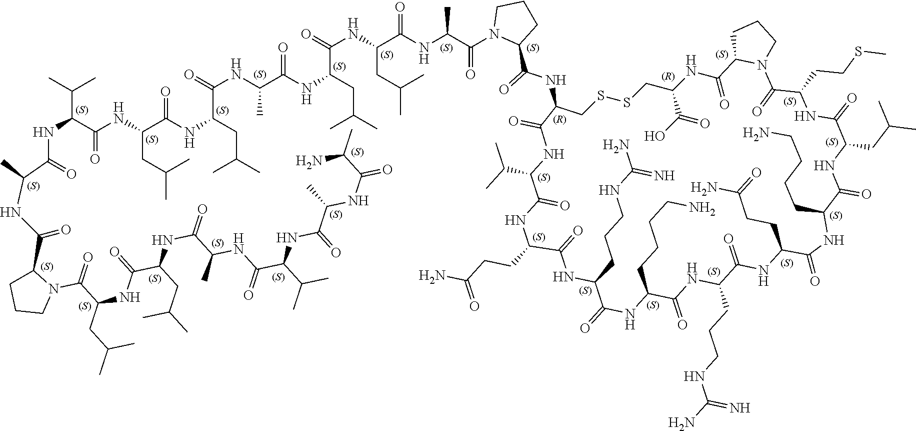

or a pharmaceutically acceptable salt thereof. In some embodiments, the compound is in a salt form. In some embodiments, the salt form of the compound includes at least one acetate counterion. In some embodiments, Compound 1 is an acetate salt of the compound.

Compound 1 may be represented by the formula:

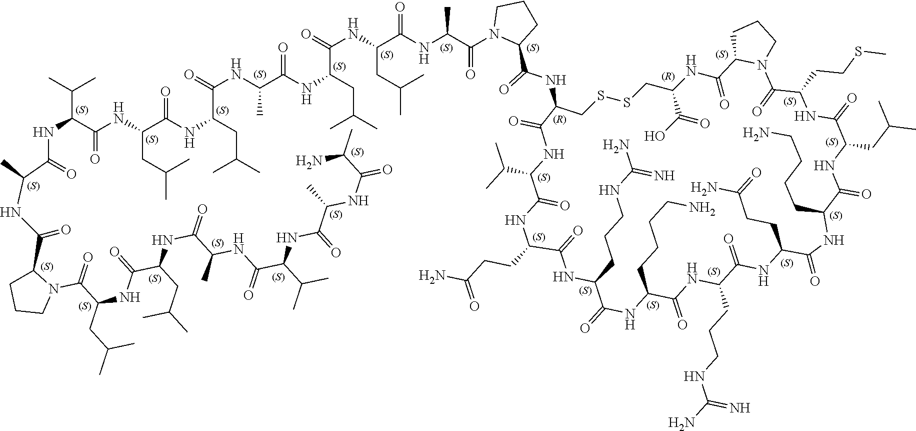

or a salt thereof.

In some embodiments, the compound comprises an amino acid sequence selected from Table 1.

TABLE 1
Leukocyte-targeting molecules.

| SEQ ID NO: | SEQUENCE |
|---|---|
| 2 | AAVAPAVX¹⁶X¹⁶AX¹⁶X¹⁶A |
| 3 | AAVALLPAVLLALLA |
| 4 | X¹X¹AAVALLPAVLLALLAX¹X¹ |
| 5 | KKAAVALLPAVLLALLAKK |
| 6 | RRAAVALLPAVLLALLARR |
| 7 | RRAAVALLPAVLLALLARK |
| 8 | RKAAVALLPAVLLALLARKY |
| 9 | AAVALLPAVLLALLAPCVQRKRQKLMPC |
| 10 | X¹X²AAX³AX⁴X⁵X¹⁷AX⁶X⁷X⁸AX⁹X¹⁰A(P)ₙ |
| | X¹¹X¹²(X¹³)ₙ |
| 11 | X¹X²AAVALLPAVLLALLAPX¹¹X¹²(X¹³)ₙ |
| 12 | X¹X²AAX³AX⁴X⁵X¹⁷AX⁶X⁷X⁸AX⁹X¹⁰APX¹¹X¹² |
| | (X¹³)ₙCVQRKRQKLMPC |
| 13 | AAVALLPAVLLALLAPVQRKRQKLMP |
| 14 | AAVALLPAVLLALLAPCVQRKRQKLMPC |
| | (cyclized by SS bond at C¹⁷ and C²⁸) |
| 15 | AAVAPAVX¹⁶X¹⁶AX¹⁶X¹⁶AP |
| 16 | AAVALLPAVLLALLAP |
| 17 | (X¹⁴)ₙ(X¹⁵)ₙAAVALLP(X¹⁴)ₘ |
| 18 | (X¹⁴)ₙ(X¹⁵)ₙAVLLALLAP(X¹⁴)ₘ |
| 20 | (X¹⁴)ₙ(X¹⁵)ₙAAVALLAAVLLALLAP(X¹⁴)ₘ |
| 21 | TTGTLLPGVLLALVVA |
| 22 | (X¹⁴)ₙ(X¹⁵)ₙTTGTLLPGVLLALVVA(X¹⁴)ₘ |
| 23 | TTGTLLPRVLLALVVA |
| 24 | (X¹⁴)ₙ(X¹⁵)ₙTTGTLLPGVLLALVVA(X¹⁴)ₘ |
| 25 | (X¹⁴)ₙ(X¹⁵)ₙTTGTLLP(X¹⁴)ₘ |
| 26 | (X¹⁴)ₙ(X¹⁵)ₙVLLALVVA(X¹⁴)ₘ |
| 28 | RAAVALLPAVLLALLAPY(X¹⁴)ₘ |
| 29 | RAAVALLPAVLLAVLAPY(X¹⁴)ₘ |
| 30 | (X¹⁴)ₙ(X¹⁵)ₙAAVALLPAVLLALLAPDVRKRQ |
| | DLEQ KM(X¹⁴)_{z}(Y)ₘ |
| 31 | (X¹⁴)ₙ(X¹⁵)ₙAAVALLPAVLLALLAP(X¹⁴)ₘ |
| 32 | (X¹⁴)ₙ(X¹⁵),AAVALLAAVLLALLAP(X¹⁴)ₘ |
| 33 | (X¹⁴)ₙ(X¹⁵),TTGTLLPGVLLALVVA(X¹⁴)ₘ |
| 34 | (X¹⁴)ₙ(X¹⁵)ₙTTGTLLPRVLLALVVA(X¹⁴)ₘ |
| 35 | (X¹⁴)ₙ(X¹⁵)ₙTTGTLLP(X¹⁴)ₘ |
| 36 | (X¹⁴)ₙ(X¹⁵)ₙGVLLALVVA(X¹⁴)ₘ |
| 37 | (X¹⁴)ₙ(X¹⁵)ₙAAVALLPAVLLALLAPCVQKRQ |
| | KLMPC |
| 38 | KKAAVALLPAVLLALLAPKK |
| 39 | RRAAVALLPAVLLALLAPRR |
| 40 | RRAAVALLPAVLLALLAPRK |
| 41 | RKAAVALLPAVLLALLAPRKY |
| 42 | KRAAVALLPKK |
| 43 | KRAVLLALLAPKK |
| 44 | KRAAVALLAAVLLALLAPKK |
| 45 | KRTTGTLLPGVLLALVVAKK |
| 46 | KRTTGTLLPGVLLALVVAKK |
| 47 | KRTTGTLLPKK |
| 48 | KRVLLALVVAKK |
| 49 | KRAAVALLPKK |
| 50 | AAVALLPAVLLALLAP |
| 51 | AAVALLPAVLLALLAPCYVQRKRQKLMPC |
| 52 | AAVALLPAVLLAVLAPCVQRKRQKLMPC |
| 53 | AAVALLPAVLLALLAPCVQRDEQKLMPC |
| 54 | AAVALLPAVLLAVLAPCVQRDEQKLMPC |

X¹, X², X¹¹, and X¹² = Lys, Arg, or Orn
X³, X⁴, X⁵, X⁶, X⁷, X⁸, X⁹, X¹⁰ = Val, Leu, Ile, or Nle
X¹³ = Tyr
X¹⁴ = Lys
X¹⁵ = Arg
X¹⁶ = Leu, Ile, or Nle
X¹⁷ = Pro or Ala
n = 0 or 1;
m = 0 or 2

In some embodiments, the peptides of Table 1 may be linear or cyclized by a disulfide bond between two cysteine residues.

In some embodiments, the leukocyte-targeting molecule is covalently linked to the pharmaceutical agent. In some embodiments a linker sequence is disposed between the leukocyte-targeting molecule and the pharmaceutical agent. In some embodiments, the leukocyte-targeting molecule and the pharmaceutical agent comprise a fusion protein.

In some embodiments, the leukocyte-targeting molecule and the pharmaceutical agent are encapsulated in a micelle or a liposome. In some embodiments, the leukocyte-targeting molecule is incorporated into a micelle or liposome such that the leukocyte-targeting molecule is available on the surface of the micelle or liposome for interaction with leukocytes.

In some embodiments, the leukocyte-targeting molecules specifically target eosinophils, basophils, neutrophils, or monocytes, or a combination thereof. In some embodiments, the leukocyte-targeting molecules do not target T-lymphocytes, B-lymphocytes, or NK cells, or a combination thereof.

In some embodiments, the formulation is a formulation selected from Table 2.

As indicated above, in some embodiments the formulations provided herein may be an ointment formulation, a cream formulation, a cold cream formulation, a gel formulation, a paste formulation, a liposomal formulation, a microemulsion formulation, or a nanoparticle formulation.

Ointments may be divided into lipophilic ointments (lipophilic active ingredients may be further incorporated), anhydrous absorption bases (subsequent addition of water results in a cream), or hydrophilic ointments (hydrophilic active ingredients may be further incorporated).

Creams may be three-phase systems including an aqueous, an oily, and an emulsifying phase. Depending on the continuous (outer) phase, a distinction may be made between hydrophilic creams (oil-in water type), lipophilic creams (water-in-oil type), and amphiphilic creams (bicontinuous creams, with oil-in-water and water-in-oil components).

‘Quasi emulsions’ may be distinguished as a special variant—also known as cold creams. These are stable, at room temperature highly viscous, lipophilic ointments in which (without the addition of emulsifiers) water droplets have been incorporated. Upon application and melting of the ointment, these droplets are released onto the skin, evaporate, and thus convey a cooling effect.

Gels represent another therapeutic option for the formulations provided herein. They consist of a matrix builder that specifies a three-dimensional structural framework (for example, starches), which-dispersed in water or oil-results in a semi-solid preparation of variable viscosity.

The term paste refers to systems that contain an insoluble (particle) component suspended in ointment/oil or cream.

Liposomes are usually aqueous phases surrounded by a lipid membrane (bilayer); their molecular arrangement can be modified (lamellar structures).

Microemulsions are Newtonian fluids that, in addition to an aqueous and oily phase, also contain an emulsifier/co-emulsifier mixture, and thus have particular thermodynamic properties.

Nanoparticles may be porous particles (for example, polymeric particles) that either absorb liquids like a sponge or encapsulate liquid droplets, which liquid may comprise the active compound.

TABLE 2
Exemplary cream formulations provided herein.

| Formulation Ingredients | F1 | F2 | F3 |
|---|---|---|---|
| Compound 1 (98.7% pure by HPLC) | 1.1% w/w | 0.33% w/w | 0.11% w/w |
| Methyl Paraben, NF | 0.16% w/w | 0.16% w/w | 0.16% w/w |
| Propyl Paraben, NF, BP, EP | 0.04% w/w | 0.04% w/w | 0.04% w/w |
| Propylene Glycol, USP | 2% w/w | 2% w/w | 2% w/w |
| Isopropyl Palmitate, NF | 2% w/w | 2% w/w | 2% w/w |
| PEG 8000, NF | 0.5% w/w | 0.5% w/w | 0.5% w/w |
| PEG 400, NF | 0.5% w/w | 0.5% w/w | 0.5% w/w |
| Carbomer 974P, NF | 0.8% w/w | 0.8% w/w | 0.8% w/w |
| Methyl Cellulose 4000, cP, USP | 0.6% w/w | 0.6% w/w | 0.6% w/w |
| Polysorbate 80, NF | 0.5% w/w | 0.5% w/w | 0.5% w/w |
| Triethanolamine, NF | 0.8% w/w | 0.8% w/w | 0.8% w/w |
| Water for Injection, USP | 91.0% w/w | 91.77% w/w | 91.99% w/w |
| Formulation Ingredients | F4 | F5 | F6 |
| Compound 1 (98.7% pure by HPLC) | 1.1% w/w | 0.33% w/w | 0.11% w/w |
| Methyl Paraben, NF | 0.08% w/w | 0.08% w/w | 0.08% w/w |
| Propyl Paraben, NF, BP, EP | 0.02% w/w | 0.02% w/w | 0.02% w/w |
| Propylene Glycol, USP | 2% w/w | 2% w/w | 2% w/w |
| Isopropyl Palmitate, NF | 2% w/w | 2% w/w | 2% w/w |
| PEG 8000, NF | 0.5% w/w | 0.5% w/w | 0.5% w/w |
| PEG 400, NF | 0.5% w/w | 0.5% w/w | 0.5% w/w |
| Carbomer 974P, NF | 0.7% w/w | 0.7% w/w | 0.7% w/w |
| Methyl Cellulose 4000, cP, USP | 0.6% w/w | 0.6% w/w | 0.6% w/w |
| Polysorbate 80, NF | 0.5% w/w | 0.5% w/w | 0.5% w/w |
| Triethanolamine, NF | 0.8% w/w | 0.8% w/w | 0.8% w/w |
| Water for Injection, USP | 91.2% w/w | 91.97% w/w | 92.19% w/w |

Alternatively to the formulations of Table 2, Compound 1 of the formulations may be replaced, in some embodiments, with a peptide compound described herein.

Articles of manufacture are described, which comprise the formulations provided herein. The articles of manufacture may include forms of the formulations suitable for administration or storage.

The present formulations and associated materials can be finished as a commercial product by the usual steps performed in the present field, for example by appropriate sterilization and packaging steps. For example, the material can be treated by UV/vis irradiation (200-500 nm), for example using photo-initiators with different absorption wavelengths (for example, Irgacure 184, 2959), preferably water-soluble initiators (for example, Irgacure 2959). Such irradiation is usually performed for an irradiation time of 1-60 min, but longer irradiation times may be applied, depending on the specific method. The material according to the present disclosure can be finally sterile-wrapped so as to retain sterility until use and packaged (for example, by the addition of specific product information leaflets) into suitable containers (boxes, etc.).

According to further embodiments, the present compounds can also be provided in kit form combined with other components necessary for administration of the material to the patient. For example, disclosed kits, such as for use in the treatment of leukocyte associated diseases, can further comprise, for example, administration materials. The kits are designed in various forms based on the specific deficiencies they are designed to treat.

The formulations provided herein may be prepared and placed in a container for storage at ambient or elevated temperature. When the formulation is stored in a polyolefin plastic container, for example, as compared to a polyvinyl chloride plastic container, discoloration of the formulation or adsorption of the compound of the formulation may be reduced. Without wishing to be bound by theory, the container may reduce exposure of the container's contents to electromagnetic radiation, whether visible light (for example, having a wavelength of about 380-780 nm) or ultraviolet (UV) light (for example, having a wavelength of about 190-320 nm (UV B light) or about 320-380 nm (UV A light)). Some containers also include the capacity to reduce exposure of the container's contents to infrared light, or a second component with such a capacity. The containers that may be used include those made from a polyolefin such as polyethylene, polypropylene, polyethylene terephthalate, polycarbonate, polymethylpentene, polybutene, or a combination thereof, especially polyethylene, polypropylene, or a combination thereof. In some embodiments, the container is a bag, tube, tub, or bottle. In some embodiments, the container is a glass container. The container may further be disposed within a second container, for example, a paper, cardboard, paperboard, metallic film, or foil, or a combination thereof, container to further reduce exposure of the container's contents to UV, visible, or infrared light. Formulations benefiting from reduced discoloration, decomposition, or both during storage, include the formulations provided herein. The compounds or compositions provided herein may need storage lasting up to, or longer than, three months; in some cases up to, or longer than one year. The containers may be in any form suitable to contain the contents; for example, a bag, a bottle, a tube, a tub, or a box, or a combination thereof.

In some embodiments, provided herein are packaged formulations, or packaged pharmaceutical formulation, for example, kits, comprising a container housing an effective amount of a compound in a formulation described herein, and instructions for using the formulation in accordance with one or more of the methods provided herein.

### Methods

In some embodiments, the formulations provided herein are useful in treating leukocyte associated diseases, immune, inflammatory, or neurodegenerative diseases on administration of the formulation (for example, topical administration) to a subject in need thereof.

In some embodiments, the formulations provided herein are useful for targeting of diagnostic, therapeutic, and prophylactic agents to leukocytes. In some embodiments, the leukocytes are targeted in a subject in need of treatment for an immune (for example, autoimmune), inflammatory, or neurodegenerative disease, or in need of reduction of aberrant cytokine, chemokine, or growth factor production or signaling.

In some embodiments, the formulations applied topically are useful in treating acute lung inflammation, a liver disease, hypercholesterolemia, atherosclerosis, fatty liver, diabetes type 1, a skin disease or disorder (for example topically induced inflammation or burn), or a sepsis (for example, polymicrobial sepsis). In some embodiments, the formulations applied topically are useful in treating alopecia areata. In some embodiments, provided herein are methods of treating an autoimmune disease in a subject in need thereof, comprising topically administering to the subject an effective amount of a formulation described herein, such as a cream formulation of Table 2. In some embodiments, the inflammatory disease refers to a disease or disorder including, but not limited to, acute disseminated encephalomyelitis (ADEM), Addison's disease, an allergy, allergic rhinitis, Alzheimer's disease, anti-phospholipid antibody syndrome (APS), an arthritis such as, for example, a monoarthritis, an oligoarthritis, or a polyarthritis like an osteoarthritis, a rheumatoid arthritis, a juvenile idiopathic arthritis, a septic arthritis, spondyloarthropathy, gout, pseudogout, Still's disease, asthma, autoimmune hemolytic anemia, autoimmune hepatitis, an autoimmune inner ear disease, bullous pemphigoid, celiac disease, Chagas disease, chronic obstructive pulmonary disease (COPD), diabetes mellitus type 1 (IDDM), endometriosis, a gastrointestinal disorder such as, for example, an irritable bowel disease or an inflammatory bowel disease like Crohn's disease or an ulcerative colitis, Goodpasture's syndrome, Graves' disease, Guillain-Barre syndrome (GBS), Hashimoto's thyroiditis, hidradenitis suppurativa, idiopathic thrombocytopenia purpura, interstitial cystitis, a lupus, such as, for example, a discoid lupus erythematosus, a drug-induced lupus erythematosus, a lupus nephritis, a neonatal lupus, a subacute cutaneous lupus erythematosus, a chronic cutaneous lupus, or a systemic lupus erythematosus, morphea, multiple sclerosis (MS), myasthenia gravis, a myopathy such as, for example, a dermatomyositis, an inclusion body myositis, or a polymyositis, myositis, narcolepsy, neuromyotonia, Parkinson's disease, pemphigus vulgaris, pernicious anaemia, primary biliary cirrhosis, psoriasis, recurrent disseminated encephalomyelitis, rheumatic fever, scleroderma, Sjögren's syndrome, a skin disorder such as, for example, dermatitis, eczema, statis dermatitis, atopic dermatitis, hidradenitis suppurativa, psoriasis, rosacea, acne, or scleroderma, tenosynovitis, uveitis, vasculitis such as, for example, Buerger's disease, cerebral vasculitis, Churg-Strauss arteritis, cryoglobulinemia, essential cryoglobulinemic vasculitis, giant cell arteritis, Golfer's vasculitis, Henoch-Schonlein purpura, hypersensitivity vasculitis, Kawasaki disease, microscopic polyarteritis/polyangiitis, polyarteritis nodosa, polymyalgia rheumatica (PMR), rheumatoid vasculitis, Takayasu arteritis, Wegener's granulomatosis, or vitiligo. In some embodiments, the formulations applied topically are useful in treating a viral disease, for example shingles, herpes simplex type 1 infection, herpes simplex type 2 infection, or severe acute respiratory syndrome coronavirus 2 (SARS-CoV-2) infection.

In some embodiments, the autoimmune disease is a skin disorder. In some embodiments, the skin disorder is psoriasis. In some embodiments, the skin disorder is atopic dermatitis. In some embodiments, the skin disorder is treated with a topical administration of a compound, composition, or leukocyte-targeting molecule disclosed herein. In some embodiments, provided herein are methods of treating atopic dermatitis in a subject in need thereof, comprising topically administering to the subject an effective amount of a formulation described herein, such as a cream formulation of Table 2.

In some embodiments, provided herein are methods of treating a neurodegenerative disease in a subject in need thereof, comprising topically administering to the subject an effective amount of a formulation described herein, such as a cream formulation of Table 2. In some embodiments, a neurodegenerative disease refers to a disease or disorder including, but not limited to, Parkinson's disease, Alzheimer's disease, multiple sclerosis, optic neuritis, stroke, CNS trauma, amyotrophic lateral sclerosis, a neuropathy, a nervous system hypoxia, a CNS toxicity, dementia, retinopathy, Huntington's disease, synucleinopathy, epilepsy, autism, and an aging-related CNS degeneration. In some embodiments, the neurodegenerative disease is Alzheimer's disease.

In some embodiments, provided herein are methods of reducing aberrant cytokine signaling in a subject in need thereof, comprising topically administering to the subject an effective amount of a formulation described herein, such as a cream formulation of Table 2. In some embodiments, the aberrant cytokine production is cytokine release syndrome (CRS). CRS is the aberrant overproduction of pro-inflammatory cytokines such as INF-γ, IL-1α, IL-1β, IL-6, IL-8, GM-CSF, M-CSF, and TNF-α resulting in high concentrations of systemic circulating cytokines.

In some embodiments, provided herein are methods of delivering an active pharmaceutical agent or compound of a formulation provided herein to a white blood cell or a CD34+ stem cell in a subject in need thereof, comprising administering a compound in a formulation provided herein to the subject. In some embodiments, the formulation is a cream formulation of Table 2. In some embodiments, delivery of the compound or active pharmaceutical agent to the white blood cell or the CD34+ stem cell occurs within about 2 hours or less from the time of administering the formulation. In some embodiments, the subject comprises one or more leukocyte associated diseases, immune, inflammatory, or neurodegenerative diseases described herein.

In some embodiments of the methods provided herein, the active pharmaceutical agent or compound in the formulation is delivered preferentially to white blood cells and CD34+ stem cells over red blood cells.

In some embodiments of these methods, the formulation may be provided in a dosage form that is suitable for local or systemic delivery by topical administration.

The formulations provided herein may be prepared according to conventional pharmaceutical practice (see, for example, (Gennaro, A. R. ed. (2000) Remington: The Science and Practice of Pharmacy (20th ed.), Lippincott Williams & Wilkins, Baltimore, Md.; Swarbrick, J. and Boylan, J. C. eds. (1988-1999) Encyclopedia of Pharmaceutical Technology, Marcel Dekker, New York). The compounds described herein may be prepared analogous to methods described in U.S. Pat. No. 8,324,148B2, the entire content of which is incorporated by reference herein.

The therapeutic methods described herein in general include administration of a therapeutically effective amount of a compound of a formulation described herein to a subject (for example, animal) in need thereof, including a mammal. In some embodiments, the subject is a human. In some embodiments, the subject is a non-human mammal. Such treatment will be suitably administered to subjects in need thereof. Determination of those subjects “in need thereof” can be made by any objective or subjective determination by a diagnostic test or opinion of a subject or health care provider.

Actual dosage levels of the active ingredients (for example, the compounds described herein), the formulations, or the pharmaceutical formulations provided herein may be varied so as to obtain an amount of the active ingredient that is effective to achieve the desired therapeutic response for a particular patient, formulation, and mode of administration, without being toxic to the patient. In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health, and prior medical history of the patient being treated, and like factors well-known in the medical arts. A medical doctor, for example, physician or veterinarian, having ordinary skill in the art may readily determine and prescribe the effective amount of the pharmaceutical formulation required. For example, the physician or veterinarian could start doses of the compounds employed in the pharmaceutical formulation at levels lower than that required in order to achieve the desired therapeutic effect and gradually increase the dosage until the desired effect is achieved.

The following examples further illustrate aspects of the present disclosure. However, they are in no way a limitation of the teachings or disclosure as described herein.

### EXAMPLES

### Example 1

Formulations of Table 2 are topically administered to a subject suffering from atopic dermatitis, whether administered by the subject or another individual, to a portion of a subject's skin. A clinical reduction in the severity of the subject's atopic dermatitis is observed.

### Example 2

Formulations of Table 2 comprising the peptide with a label (for example, the compound is labeled with one or more ³H radiolabel or a fluorescein (FITC) label) are topically administered to a subject suffering from a leukocyte associated disease, whether administered by the subject or another individual, to a portion of a subject's skin. Whole blood is obtained from the subject, and the various components of the blood are separated and analyzed by flow cytometry.

Panels of monoclonal antibodies-labeled with fluorescent dyes are used to phenotypically identify leukocyte cells penetrated by a compound of the formulations, including classical monocytes, non-classical monocytes, neutrophils, basophils, eosinophils, T-lymphocytes, B-lymphocytes, and NK cells. Classical monocytes are characterized by high level expression of the CD14 cell surface receptor (CD14++ CD16− monocyte). Non-classical monocytes show low level expression of CD14 and additional co-expression of the CD16 receptor (CD14+CD16++ monocyte). The compound of the formulation administered is observed by its fluorescence signature to preferentially reside in leukocyte cells over red blood cells.

### Example 3. A 7-Day Repeat Dose Tolerance Dermal Study of Compound 1 in Gottingen Mini-Pigs (Non-GLP)

The purpose of this study was to evaluate the dermal dose tolerance at the maximum feasible dose (concentration and application rate) of Compound 1 in Formulation 1, Formulation 2, or Formulation 3 of Table 2, including placebo (F1, F2, or F3 without Compound 1) when applied by topical dermal application to Gottingen Mini-Pigs twice daily for seven days.

Three experimentally naive male Gottingen Mini-Pigs, 4-5 months old and weighing 10.6-13.8 kilograms at the outset of the study had four dose sites (100 cm² per site) numbered 1, 2, 3, and 4. Each formulation was assigned to a dose site for the duration of the study, as shown in Table 3 below. The dose sites were not rotated.

TABLE 3

| | Concentration | Application |
|---|---|---|
| Site Number/Treatment | (% w/w) | Volume (g) |
| 1. Placebo | 0.0 | 0.75 |
| 2. Low Concentration | 0.11 (Formulation 3) | 0.75 |
| 3. Medium Concentration | 0.33 (Formulation 2) | 0.75 |
| 4. High Concentration | 1.1 (Formulation 1) | 0.75 |

Animals were dosed with all formulations twice daily (two doses per site per day; 6 hours+/−30 minutes between the first and second dose for 7 days). Mortality/morbidity was performed twice daily and once on the day of scheduled euthanasia. On Days 1-7 animals were observed prior to dosing and at 1-2 hours post-dose. A final observation was recorded on Day 8 prior to scheduled euthanasia. Body weights were recorded on Days 1 and 7. Food consumption was determined daily. Blood for toxicokinetic evaluation was collected on Days 1 and 7 at 0.5, 1, 2, 4, 6, 8 and 24 hours post-dose. Plasma samples were assayed, and the results showed that at all time points, the amount of Compound 1 was below the Quantifiable Limit (<1.00 ng/ml). Animals were sacrificed on Day 8 and a gross necropsy was performed. The dosing area (skin) was collected and preserved.

There were no early deaths during the study, no clinical signs or dermal effects noted as all animals appeared normal throughout the study, no definite test article-related effects on body weights during the study, no test article-related effects on food consumption during the study, and no drug-related gross necropsy findings. Test article application sites in all animals appeared normal.

In conclusion, male Gottingen mini-pigs tolerated Compound 1 at concentrations of 0, 0.11, 0.33, and 1.1% w/w in the formulations tested when applied topically to the skin twice daily for 7 consecutive days. There were no signs of dermal irritation and no general signs of toxicity. No test article related effects were observed on body weights or food consumption. At necropsy on Day 8, all tissues appeared to be macroscopically normal. The no-observed-adverse-effect level (NOAEL) for Compound 1 was determined to be the highest dose tested: 0.75 grams of 1.1% w/w formulation applied twice daily for 7 days.

### Example 4. Phorbol Myristoyl Acetate (PMA)-Induced Skin Inflammatory Mouse Model (Irritant Contact Dermatitis)

The anti-inflammatory efficacy of Compound 1 on a Phorbol Myristoyl Acetate (PMA)-induced skin inflammatory mouse model (irritant contact dermatitis) is shown following topical application of a formulation including Compound 1 herein. The skin biopsies in the study demonstrate increased inflammatory cell infiltration in response to PMA, a known inducer of pro-inflammatory signaling pathways to the cell's nucleus, is reduced by dose-dependent Compound 1 treatment. The ear thickness measurements of intact ears show that ear swelling due to increased microvascular permeability induced by PMA is attenuated by topical Compound 1 treatment. Ear punch biopsies show that increased swelling and cellular infiltration induced by PMA are reduced by Compound 1 treatment 8 h post-challenge. Furthermore, the results demonstrate that continuous Compound 1 treatment (for example, repeated daily treatment) does not impede skin wound healing and hair re-growth after repeated surgical trauma.

### Example 5. PK/Biodistribution Following Single Topical Dose of Compound 1

Whole blood and plasma radioactivity analysis, quantitative whole-body autoradiography (QWBA), and micro-autoradiography (mARG) studies are performed following a single topical dose administration to male rats. Formulation 1, Formulation 2, or Formulation 3, where Compound 1 is 3H-Compound 1, are applied in a single topical dose at a target dose of 5 mg/kg to six male albino rats.

An additional three male albino rats received a single topical appropriate amount of CETAPHIL (R) applied to the dose site approximately 1 hour prior to application of ³H-Compound 1 in a single topical dose at a target dose of 5 mg/kg. An individual rat receiving the emollient pre-treatment was used for a single time-point of 2 hours, 8 hours, and 48 hours. The 48 hour time-point rat had removed its dosage site covering about 33-45 hours after dose administration, and may have resulted in an oral administration of the treatment by grooming.

PK data demonstrate that 3H-Compound 1 applied in a cream formulation can penetrate deep enough into the skin to reach blood vessels in less than one hour after a single topical administration. The PK data show that ³H-Compound 1 partitions to the WBCs (white blood cells) of blood preferentially over, for example, RBCs and RBC/WBC-depleted plasma. 3H-Compound 1 detected in the blood reaches a Tₘₐₓ at 8 hours and persists for longer than 48 hours after a single administration—T_{1/2} is not reached at 48 hours, the longest time-point taken. Results are shown in Table 4, Table 5, and Table 6.

TABLE 4
Concentrations of radioactivity in whole blood, plasma, white blood
cell depleted blood and recovered white blood cells (expressed as
ng equivalents/mL) following a single topical administration of ³H-Compound
1 to male albino rats at a target dose of 5 mg/kg.

| | ³H-Compound 1 ng equivalents/mL¹ | | | | | |
|---|---|---|---|---|---|---|
| Time post-dose (hours) | 1 h | 2 h | 4 h | 8 h | 24 h | 48 h |
| Whole Blood | 1.923 | 3.850 | 4.303 | 11.68 | 5.593 | 4.978 |
| Plasma | 1.764 | 4.818 | 4.896 | 14.53 | 6.709 | 6.287 |
| WBC depleted blood | BLQ | BLQ | 0.735 | 2.282 | 1.166 | 1.109 |
| Recovered White Blood Cells² | BLQ | BLQ | BLQ | BLQ | BLQ | BLQ |

¹Assume 1 g blood/plasma is equivalent to 1 mL
²Reverse wash of filters - may not represent total white blood cells
BLQ: Below limit of accurate quantification

TABLE 5
Blood partition calculation following single topical administration
of ³H-Compound 1 to male albino rats at a target dose of 5 mg/kg.

| | | | | | | | |
|---|---|---|---|---|---|---|---|
| | | | | | | | WBC |
| Time | Blood | Plasma | Blood:plasma | % cell | Partition | Non-plasma | depleted |
| (hours) | (ng/mL) | (ng/mL) | ratio | volume | coefficient | compartment | blood |

single topical administration of ³H-Compound 1 without CETAPHIL(R) emollient
(³H-Compound 1 ng equivalents/mL¹)

| | | | | | | | |
|---|---|---|---|---|---|---|---|
| 1 | 1.923 | 1.764 | 1.090 | 0.38 | 56.9 | 43.1 | BLQ |
| 2 | 3.850 | 4.818 | 0.799 | 0.40 | 75.1 | 24.9 | BLQ |
| 4 | 4.303 | 4.896 | 0.879 | 0.42 | 66.0 | 34.0 | 0.735 |
| 8 | 11.68 | 14.53 | 0.804 | 0.42 | 72.2 | 27.8 | 2.282 |
| 24 | 5.593 | 6.709 | 0.834 | 0.42 | 69.6 | 30.4 | 1.166 |
| 48 | 4.978 | 6.287 | 0.792 | 0.38 | 78.3 | 21.7 | 1.109 |

CETAPHIL(R) emollient applied 1 hr prior to single topical administration of ³H-Compound 1
(³H-Compound 1 ng equivalents/mL¹)

| | | | | | | | |
|---|---|---|---|---|---|---|---|
| 2 | 4.444 | 5.443 | 0.816 | 0.40 | 73.5 | 26.5 | 2.333 |
| 8 | 16.05 | 21.04 | 0.763 | 0.40 | 78.7 | 21.3 | 3.223 |
| 482 | 1630 | 2312 | 0.705 | 0.43 | 80.8 | 19.2 | 303.9 |

¹Assume 1 g blood/plasma is equivalent to 1 mL
²Dosing device removed by rat between ca. 33 and 45 hours after dose administration. Potential grooming of the dose site may have caused an oral administration of ³H-Compound 1.
BLQ: Below limit of accurate quantification
WBC

TABLE 6
Pharmacokinetic parameters for total radioactivity
in blood, plasma and WBCD blood following single topical
administration of ³H-Compound 1 to male albino
rats at a target dose of 5 mg/kg.

| Parameter | Plasma | Whole blood | Whole blood -WBC |
|---|---|---|---|
| Tmax (hours) | 8.000 | 8.000 | 8.000 |
| Cₘₐₓ¹ (ng/mL) | 14.53 | 2.282 | 11.68 |
| AUC₀₋ₜ (h · ng/mL) | 370.6 | 61.39 | 302.9 |

Tₘₐₓ: Time at which maximum concentration was apparent
Cₘₐₓ: Maximum measured concentration
AUC₀₋ₜ: Area under the curve from 0 to last quantifiable data point
WBC: White blood cells
¹Insufficient data points after Cₘₐₓ to calculate further PK parameters

### Example 6. Phase 1/2a Study of 1.1% Compound 1 (Formulation 4) Open-Label, Body-Surface Area, Dose-Escalation Safety and Exploratory Efficacy Trial in Mild to Moderate Atopic Dermatitis (AD) Patients

26 subjects having mild to moderate atopic dermatitis and about 3-70% treatable body surface area (BSA) were enrolled in the study. Subjects were treated at all treatable AD affected areas twice a day (BID) for 7 days with 1.1% Compound 1 (as Formulation 4), and had a 2-week follow-up assessment after end of treatment (EOT). Dosing was targeted at 0.62 g/kg/day of the formulation or 6.82 mg/kg/day of Compound 1. Based on an average body weight of 60 kg, and average BSA of 1.62 m2, a recommended starting dose of 2.5 g of a formulation (for example, Formulation 1 or 4; Formulations 2, 3, 5, and 6 may be dosed higher due to the lower concentration of Compound 1) is proposed as a starting point. Severity of atopic dermatitis (vIGA-AD score), treatable BSA, and the following 11 signs and symptoms were assessed: dryness, erythema, burning/stinging, erosion/ulceration, edema/swelling, itching, pain treated area, crusting, vesiculation, flaking/scaling, and bleeding. Results are shown below in Table 7.

26/26 mild and moderate AD patients were enrolled and 23 fully completed treatment at 4 sites. Compound 1 has been well tolerated; there have been no adverse events (AEs); 3 noncompliant subjects (one of which did one day of treatment which lowered their vIGA-AD and treatable BSA scores). 24 of 26 patients responded to Compound 1 in terms of either a reduction in size of treatable BSA (20-100%) or decrease in VIGA-AD severity score, or both, demonstrating biologic activity at end of treatment (EOT) or at 2-week follow-up visit. 57% of subjects at EOT (BID for 7 days) had either 0-clear or 1-almost clear vIGA-AD scores; 61% had 0-clear or 1-almost clear skin at the 2-week post treatment. At EOT 6 subjects, or 26%, had improved vIGA-AD scores of 2 units; at 2-week follow up, 10 subjects or 44% had an improvement in vIGA-AD scores of 2 units; only 3 (13%) subjects at EOT had a vIGA-AD score that worsened 1 unit at 2-week follow up and 0 worsened 2 units at 2-week follow up. Patient diaries were scored for 11 signs and symptoms of AD resulting in a net negative score (overall signs and symptoms were reduced; all but one scored by physician).

TABLE 7
Phase 1/2a study results.

| Clinical Site | | | | | Screening | |
|---|---|---|---|---|---|---|
| 2 | 3 | 4 | 5 | Patient No. | vIGA-AD | BSA (%) |
| X | | | | 2-001 | 2-mild | 4 |
| X | | | | 2-002 | 2-mild | 5 |
| X | | | | 2-003 | 2-mild | 5 |
| X | | | | 2-004 | 2-mild | 5 |
| X | | | | 2-005 | 2-mild | 5 |
| X | | | | 2-008 | 2-mild | 7 |
| X | | | | 2-009 | 2-mild | 8 |
| X | | | | 2-010 | 2-mild | 7 |
| | X | | | 3-001 | 2-mild | 7.25 |
| | X | | | 3-002 | 2-mild | 10 |
| X | | | | 2-011 | 3-moderate | 15 |
| X | | | | 2-012 | 3-moderate | 20 |
| X | | | | 2-014 | 3-moderate | 15 |
| | | X | | 4-001 | 3-moderate | 24 |
| | | | X | 5-001 | 2-mild | 14 |
| | | | X | 5-002 | 3-moderate | 28 |
| | | | X | 5-004 | 3-moderate | 30 |
| | | X | | 4-002 | 2-mild | 25 |
| | | X | | 4-003 | 3-moderate | 25 |
| X | | | | 2-016 | 3-moderate | 50 |
| | | | X | 5-007 | 3-moderate | 54 |
| | | | X | 5-008 | 3-moderate | 50 |
| | | | X | 5-009 | 3-moderate | 62 |
| | | | X | 5-010 | 3-moderate | 50 |
| | | X | | 4-004 | 3-moderate | 49 |
| X | | | | 2-019 | 3-moderate | 50 |

| | Baseline | | End of Treatment | | 2-Week Follow Up |
|---|---|---|---|---|---|
| Patient No. | vIGA-AD | BSA (%) | vIGA-AD | BSA (%) | vIGA-AD (BSA %) |
| 2-001 | 2-mild | 3 | 2-mild | 2 | 2-mild |
| 2-002 | 2-mild | 5 | 1-almost clear | 4 | 2-mild |
| 2-003 | 2-mild | 5 | 1-almost clear | 3 | 0-clear |
| 2-004 | 2-mild | 5 | 0-clear | 0 | 0-clear |
| 2-005 | 2-mild | 5 | 1-almost clear | 3 | 0-clear |
| 2-008 | 2-mild | 7 | 0-clear | 0 | 1-almost clear |
| 2-009 | 2-mild | 8 | 1-almost clear | 4 | 1-almost clear |
| 2-010 | 2-mild | 7 | 1-almost clear | 2 | 1-almost clear |
| 3-001 | 3-moderate | 7 | 3-moderate | 7 | 2-mild |
| 3-002 | 2-mild | 10 | 1-almost clear | 10 | 1-almost clear |
| 2-011 | 3-moderate | 15 | 1-almost clear | 5 | 1-almost clear |
| 2-012 | 3-moderate | 20 | 3-moderate | 10 | 2-mild |
| 2-014 | 3-moderate | 15 | 0-clear | 0 | 1-almost clear |
| 4-001 | 3-moderate | 21 | 3-moderate | 24 | 2-mild |
| 5-001 | 2-mild | 14 | 1-almost clear | 12 | 0-clear |
| 5-002 | 3-moderate | 28 | 1-almost clear | 12 | 1-almost clear (10%) |
| 5-004 | 3-moderate | 30 | 2-mild | 30 | 3-moderate (30%) |
| 4-002 | 2-mild | 25 | 2-mild | 11 | 2-mild (17%) |
| 4-003 | 3-moderate | 25 | 3-moderate | 17 | 2-mild (7%) |
| 2-016 | 3-moderate | 50 | 2-mild | 35 | 1-almost clear (35%) |
| 5-007 | 3-moderate | 54 | 3-moderate | 54 | 3-moderate (54%) |
| 5-008 | 3-moderate | 50 | 3-moderate | 50 | 3-moderate (50%) |
| 5-009 | 3-moderate | 62 | 3-moderate | 62 | 3-moderate (62%) |
| 5-010 | 3-moderate | 50 | 3-moderate | 50 | 3-moderate (50%) |
| 4-004 | 3-moderate | 49 | 1-almost clear | 7 | 1-almost clear (5%) |
| 2-019 | 3-moderate | 55 | 2-mild | 35 | 1-almost clear (5%) |

| | |
|---|---|
| vIGA-AD | Validated InvestigatorGlobal Assessment |
| | for Atopic Dermatitis (Lilly) |
| BSA | Treatable Body Surface Area* Affected |
| | by Atopic Dermatitis |
| SF | Screen Failure: Fails to Continue to |
| | Meet Inclusion Criteria |
| EOT | End of Treatment |
| Clinical Site marked by X | (*Note: palm of hand with fingers |
| | closed is ~1% BSA) |
| Cohort 1: 3-6% BSA | 5 subjects |
| Cohort 2: 6-12% BSA | 5 subjects |
| Cohort 3: 12-24% BSA | 5 subjects |
| Cohort 4: 24-48% BSA | 4 subjects |
| Cohort 5: 48-70% BSA | 7 subjects |
| Enrolled and/or Treated Total | 26 subjects |

We claim:

1. A formulation, comprising about 0.05 to about 2.0% w/w of a compound comprising an amino acid sequence of a formula selected from:
(SEQ ID NO: 9)
AAVALLPAVLLALLAPCVQRKRQKLMPC;
(SEQ ID NO: 2)
AAVAPAVX¹⁶X¹⁶AX¹⁶X¹⁶A;
(SEQ ID NO: 3)
AAVALLPAVLLALLA;
(SEQ ID NO: 4)
X¹X¹AAVALLPAVLLALLAX¹X^{1;}
(SEQ ID NO: 5)
KKAAVALLPAVLLALLAKK;
(SEQ ID NO: 6)
RRAAVALLPAVLLALLARR;
(SEQ ID NO: 7)
RRAAVALLPAVLLALLARK;
(SEQ ID NO: 8)
RKAAVALLPAVLLALLARKY;
(SEQ ID NO: 10)
X¹X²AAX³AX⁴X⁵X¹⁷AX⁶X⁷X⁸AX⁹X¹⁰A(P)ₙX¹¹X¹²(X¹³)_{n;}
(SEQ ID NO: 11)
X¹X²AAVALLPAVLLALLAPX¹¹X¹²(X¹³)_{n;}
(SEQ ID NO: 12)
X¹X²AAX³AX⁴X⁵X¹⁷AX⁶X⁷X⁸AX⁹X¹⁰APX¹¹X¹²(X¹³),
CVQRKRQKLMPC;
(SEQ ID NO: 13)
AAVALLPAVLLALLAPVQRKRQKLMP;
(SEQ ID NO: 14)
AAVALLPAVLLALLAPCVQRKRQKLMPC;
(SEQ ID NO: 15)
AAVAPAVX¹⁶X¹⁶AX¹⁶X¹⁶AP;
(SEQ ID NO: 16)
AAVALLPAVLLALLAP;
(SEQ ID NO: 17)
(X¹⁴)ₙ(X¹⁵)ₙAAVALLP(X¹⁴)_{m;}
(SEQ ID NO: 18)
(X¹⁴)ₙ(X¹⁵)ₙAVLLALLAP(X¹⁴)_{m;}
(SEQ ID NO: 20)
(X¹⁴)ₙ(X¹⁵)ₙAAVALLAAVLLALLAP(X¹⁴)_{m;}
(SEQ ID NO: 21)
TTGTLLPGVLLALVVA;
(SEQ ID NO: 22)
(X¹⁴)ₙ(X¹⁵)ₙTTGTLLPGVLLALVVA(X¹⁴)_{m;}
(SEQ ID NO: 23)
TTGTLLPRVLLALVVA;
(SEQ ID NO: 24)
(X¹⁴)ₙ(X¹⁵)ₙTTGTLLPGVLLALVVA(X¹⁴)_{m;}
(SEQ ID NO: 25)
(X¹⁴)ₙ(X¹⁵)ₙTTGTLLP(X¹⁴)_{m;}
(SEQ ID NO: 26)
(X¹⁴)ₙ(X¹⁵)ₙVLLALVVA(X¹⁴)_{m;}
(SEQ ID NO: 28)
RAAVALLPAVLLALLAPY(X¹⁴)_{m;}
(SEQ ID NO: 29)
RAAVALLPAVLLAVLAPY(X¹⁴)_{m;}
(SEQ ID NO: 30)
(X¹⁴)ₙ(X¹⁵)ₙAAVALLPAVLLALLAPDVRKRQDLEQKM(X¹⁴)_{z}(Y)_{m;}
(SEQ ID NO: 31)
(X¹⁴)ₙ(X¹⁵)ₙAAVALLPAVLLALLAP(X¹⁴)_{m;}
(SEQ ID NO: 32)
(X¹⁴)ₙ(X¹⁵)ₙAAVALLAAVLLALLAP(X¹⁴)_{m;}
(SEQ ID NO: 33)
(X¹⁴)ₙ(X¹⁵)ₙTTGTLLPGVLLALVVA(X¹⁴)_{m;}
(SEQ ID NO: 34)
(X¹⁴)ₙ(X¹⁵)ₙTTGTLLPRVLLALVVA(X¹⁴)_{m;}
(SEQ ID NO: 35)
(X¹⁴)ₙ(X¹⁵)ₙTTGTLLP(X¹⁴)_{m;}
(SEQ ID NO: 36)
(X¹⁴)ₙ(X¹⁵)ₙGVLLALVVA(X¹⁴)_{m;}
(SEQ ID NO: 37)
(X¹⁴)ₙ(X¹⁵)ₙAAVALLPAVLLALLAPCVQKRQKLMPC;
(SEQ ID NO: 38)
KKAAVALLPAVLLALLAPKK;
(SEQ ID NO: 39)
RRAAVALLPAVLLALLAPRR;
(SEQ ID NO: 40)
RRAAVALLPAVLLALLAPRK;
(SEQ ID NO: 41)
RKAAVALLPAVLLALLAPRKY;
(SEQ ID NO: 42)
KRAAVALLPKK;
(SEQ ID NO: 43)
KRAVLLALLAPKK;
(SEQ ID NO: 44)
KRAAVALLAAVLLALLAPKK;
(SEQ ID NO: 45)
KRTTGTLLPGVLLALVVAKK;
(SEQ ID NO: 46)
KRTTGTLLPGVLLALVVAKK;
(SEQ ID NO: 47)
KRTTGTLLPKK;
(SEQ ID NO: 48)
KRVLLALVVAKK;
(SEQ ID NO: 49)
KRAAVALLPKK;
(SEQ ID NO: 50)
AAVALLPAVLLALLAP;
(SEQ ID NO: 51)
AAVALLPAVLLALLAPCYVQRKRQKLMPC;
(SEQ ID NO: 52)
AAVALLPAVLLAVLAPCVQRKRQKLMPC;
(SEQ ID NO: 53)
AAVALLPAVLLALLAPCVQRDEQKLMPC;
or
(SEQ ID NO: 54)
AAVALLPAVLLAVLAPCVQRDEQKLMPC;
or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
X¹⁴ is lysine;
X¹⁵ is arginine;
X¹⁶ is leucine, isoleucine, or norleucine;
X¹⁷ is proline or alanine;
n is 0 or 1; and
m is 0 or 2.

2. A formulation, comprising about 0.05 to about 2.0% w/w of a compound comprising an amino acid sequence of the formula:
(SEQ ID NO: 55)
X¹X²AAX³AX⁴X⁵PAX⁶X⁷X⁸AX⁹X¹⁰A(P)ₙX¹¹X¹²(X¹³)ₘ
or a pharmaceutically acceptable salt thereof;
wherein
X¹, X², X¹¹, and X¹² are each, independently, lysine, arginine, or ornithine;
X³, X⁴, X⁵, X⁶, X⁷, X⁸, X⁹, and X¹⁰ are each, independently, valine, leucine, isoleucine, or norleucine;
X¹³ is tyrosine;
m is 0, 1, or 2; and
n is 0 or 1.

3. The formulation of claim 1 or claim 2, wherein X³ and X⁶ are valine.

4. The formulation of one of claims 1-3, wherein X⁴, X⁵, X⁷, X⁸, X⁹, and X¹⁰ are each, independently, leucine, isoleucine, or norleucine.

5. The formulation of claim 2, wherein the compound comprises an amino acid sequence of the formula:
X¹X²AAVALLPAVLLALLA(P)ₘX¹¹X¹²(X¹³)ₙ
or a pharmaceutically acceptable salt thereof.

6. The formulation of one of claims 1-5, wherein the compound comprises at least one ornithine, isoleucine, or norleucine.

7. The formulation of claim 2, wherein the compound comprises an amino acid sequence selected from KKAAVALLPAVLLALLAKK, RRAAVALLPAVLLALLARR, RRAAVALLPAVLLALLARK, RKAAVALLPAVLLALLARKY, KKAAVALLPAVLLALLAPKK, RRAAVALLPAVLLALLAPRR, RRAAVALLPAVLLALLAPRK, or RKAAVALLPAVLLALLAPRKY.

8. The formulation of one of claims 1-7, wherein the compound further comprises the amino acid sequence CVQRKRQKLMPC.

9. The formulation of claim 2, wherein the compound comprises an amino acid sequence of the formula:
X¹X²AAX³AX⁴X⁵PAX⁶X⁷X⁸AX⁹X¹⁰A(P)ₘX¹¹X¹²(X¹³)ₙCVQRKRQKLMPC
or a pharmaceutically acceptable salt thereof.

10. The formulation of one of claims 1-9, wherein the compound further comprises at least one iodine.

11. The formulation of claim 9, wherein the at least one iodine is selected, independently, from ¹²³I, ¹²⁴I, ¹²⁵I, or ¹³¹I.

12. The formulation of one of claims 1-11, wherein the compound comprises at least one D-amino acid.

13. The formulation of one of claims 1-12, wherein the compound comprises an amino acid sequence AAVALLPAVLLALLACVQRKRQKLMPC.

14. The formulation of one of claims 1-12, wherein the compound comprises an amino acid sequence AAVALLPAVLLALLAPCVQRKRQKLMPC.

15. The formulation of one of claims 1-14, wherein the compound comprises a cyclic peptide.

16. The formulation of claim 13 or claim 14, wherein C¹⁶ and C²⁷ or C¹⁷ and C²⁸ are covalently linked.

17. The formulation of one of claims 1-16, wherein the compound comprises two cysteines that are covalently linked by a disulfide bond.

18. The formulation of one of claims 1-17, wherein the compound is covalently linked to an active pharmaceutical agent.

19. The formulation of one of claims 1-12, wherein the compound comprises 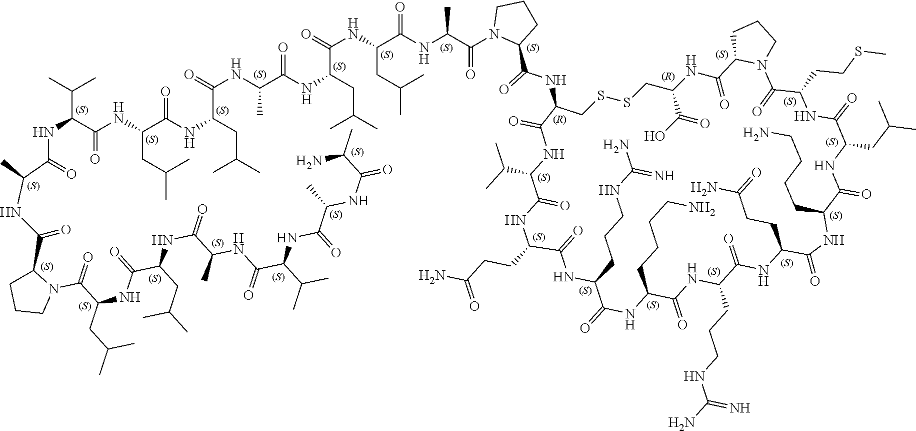
or a pharmaceutically acceptable salt thereof.

20. The formulation of one of claims 1-19, comprising about 0.11% w/w, 0.33% w/w, or 1.1% w/w of the compound.

21. The formulation of one of claims 1-20, wherein the compound is stable (for example, the compound is not more than about 1% less pure over the stated time period) for up to three months at about 20-25° C.

22. The formulation of one of claims 1-21, housed in a container that protects the formulation from exposure to direct light.

23. The formulation of one of claims 1-22, which is an ointment formulation, a cream formulation, a cold cream formulation, a gel formulation, a paste formulation, a liposomal formulation, a microemulsion formulation, or a nanoparticle formulation.

24. The formulation of one of claims 1-22, which is a cream formulation.

25. The formulation of one of claims 1-24, further comprising one or more of propylene glycol, methylparaben, propylparaben, isopropyl palmitate, polyethylene glycol, polyvinyl carboxy polymer, methylcellulose, polyoxyethylene (20) sorbitan monooleate, triethanolamine, or water.

26. The formulation of claim 1, which is a cream formulation comprising:
about 1.1% w/w of 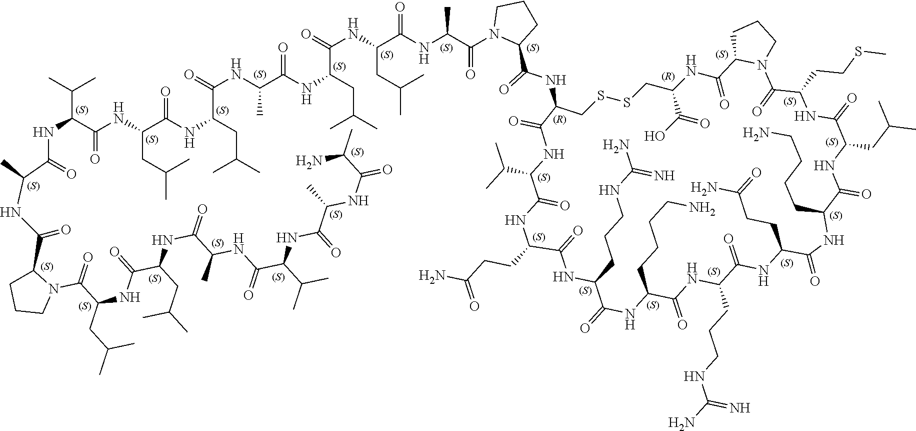
or a salt thereof;
about 0.16% w/w methyl paraben;
about 0.04% w/w propyl paraben;
about 2% w/w propylene glycol;
about 2% w/w isopropyl palmitate;
about 0.5% w/w PEG 8000;
about 0.5% w/w PEG 400;
about 0.8% w/w carbomer 974P;
about 0.6% w/w methyl cellulose 4000 cP;
about 0.5% w/w polysorbate 80;
about 0.8% w/w triethanolamine; and
about 91.0% w/w water.

27. The formulation of claim 1, which is a cream formulation comprising:
about 0.33% w/w of 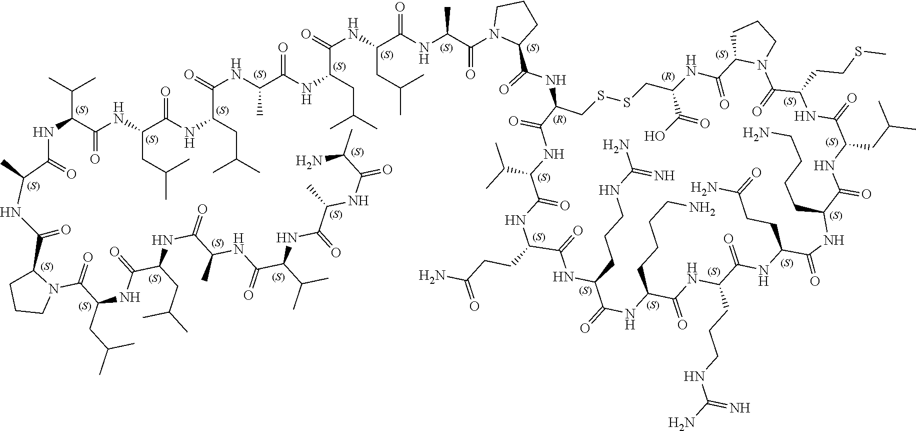
or a salt thereof;
about 0.16% w/w methyl paraben;
about 0.04% w/w propyl paraben;
about 2% w/w propylene glycol;
about 2% w/w isopropyl palmitate;
about 0.5% w/w PEG 8000;
about 0.5% w/w PEG 400;
about 0.8% w/w carbomer 974P;
about 0.6% w/w methyl cellulose 4000 cP;
about 0.5% w/w polysorbate 80;
about 0.8% w/w triethanolamine; and
about 91.77% w/w water.

28. The formulation of claim 1, which is a cream formulation comprising:
about 0.11% w/w of 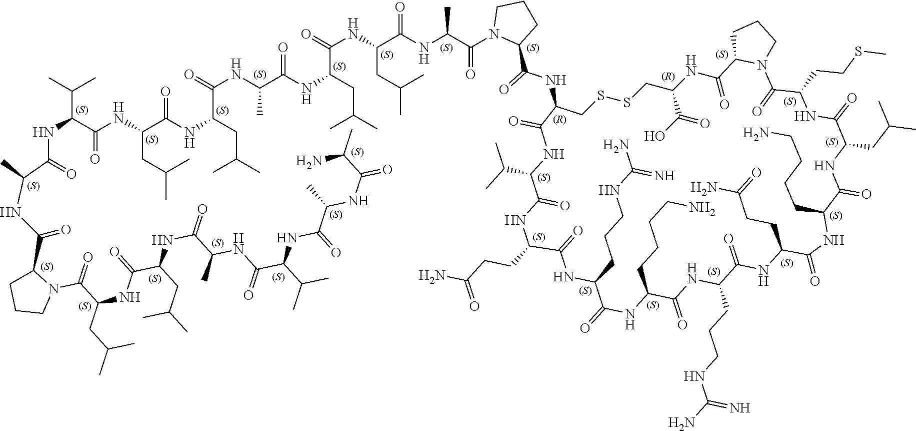
or a salt thereof;
about 0.16% w/w methyl paraben;
about 0.04% w/w propyl paraben;
about 2% w/w propylene glycol;
about 2% w/w isopropyl palmitate;
about 0.5% w/w PEG 8000;
about 0.5% w/w PEG 400;
about 0.8% w/w carbomer 974P;
about 0.6% w/w methyl cellulose 4000 cP;
about 0.5% w/w polysorbate 80;
about 0.8% w/w triethanolamine; and
about 91.99% w/w water.

29. A formulation, comprising:
about 0.05-0.2% w/w methyl paraben;
about 0.01-0.05% w/w propyl paraben;
about 1.5-2.5% w/w propylene glycol;
about 1.5-2.5% w/w isopropyl palmitate;
about 0.4-0.6% w/w PEG 8000;
about 0.4-0.6% w/w PEG 400;
about 0.5-1.0% w/w carbomer;
about 0.5-0.7% w/w methyl cellulose;
about 0.4-0.6% w/w polysorbate 80;
about 0.7-0.9% w/w triethanolamine; and
at least 87% w/w water.

30. The formulation of claim 29, comprising:
about 0.05-0.2% w/w methyl paraben;
about 0.01-0.05% w/w propyl paraben;
about 2% w/w propylene glycol;
about 2% w/w isopropyl palmitate;
about 0.5% w/w PEG 8000;
about 0.5% w/w PEG 400;
about 0.7-0.8% w/w carbomer;
about 0.6% w/w methyl cellulose;
about 0.5% w/w polysorbate 80;
about 0.8% w/w triethanolamine; and
at least 87% w/w water.

31. The formulation of claim 29 or claim 30, comprising about 87±0.5, about 88±0.5, about 89±0.5, about 90±0.5, about 91±0.5, about 92±0.5, about 93±0.5, or about 94±0.5 w/w water.

32. The formulation of one of claims 29-31, further comprising about 0.05-1.5% w/w of an active pharmaceutical agent.

33. A method of treating a leukocyte associated disease in a subject in need thereof, comprising administration of an effective amount of the formulation of one of claims 1-32 to the subject.

34. A method of treating an inflammatory or autoimmune disease in a subject in need thereof, comprising administration of an effective amount of the formulation of one of claims 1-32 to the subject.

35. A method of treating a skin disorder in a subject in need thereof, comprising administration of an effective amount of the formulation of one of claims 1-32 to the subject.

36. A method of treating atopic dermatitis in a subject in need thereof, comprising administration of an effective amount of the formulation of one of claims 1-32 to the subject.

37. A method of treating a neurodegenerative disease in a subject in need thereof, comprising administration of an effective amount of the formulation of one of claims 1-32 to the subject.

38. A method of reducing aberrant cytokine, chemokine, or growth factor signaling in a subject in need thereof, comprising administration of an effective amount of the formulation of one of claims 1-32 to the subject.

39. A method of delivering the compound of the formulation of one of claims 1-32 to a white blood cell or a CD34+ stem cell in a subject in need thereof, comprising administration of an effective amount of the formulation to the subject.

40. The method of one of claims 33-39, wherein the administration is topical administration.


SEQUENCE LISTING (55 sequences)

SEQ ID NO: 1 - protein, 14 aa, Homo sapiens
AVALLPAVLLALLA

SEQ ID NO: 2 - protein, 13 aa, synthetic construct
  VARIANT 8..9: note = X=leucine, isoleucine, or norleucine
  VARIANT 11..12: note = X=leucine, isoleucine, or norleucine
AAVAPAVXXAXXA

SEQ ID NO: 3 - protein, 15 aa, synthetic construct
AAVALLPAVLLALLA

SEQ ID NO: 4 - protein, 19 aa, synthetic construct
  VARIANT 1..2: note = X=lysine, arginine, or ornithine
  VARIANT 18..19: note = X=lysine, arginine, or ornithine
XXAAVALLPAVLLALLAXX

SEQ ID NO: 5 - protein, 19 aa, synthetic construct
KKAAVALLPAVLLALLAKK

SEQ ID NO: 6 - protein, 19 aa, synthetic construct
RRAAVALLPAVLLALLARR

SEQ ID NO: 7 - protein, 19 aa, synthetic construct
RRAAVALLPAVLLALLARK

SEQ ID NO: 8 - protein, 20 aa, synthetic construct
RKAAVALLPAVLLALLARKY

SEQ ID NO: 9 - protein, 28 aa, synthetic construct
AAVALLPAVLLALLAPCVQRKRQKLMPC

SEQ ID NO: 10 - protein, 21 aa, synthetic construct
  VARIANT 1..2: note = X=lysine, arginine, or ornithine
  VARIANT 5: note = X=valine, leucine, isoleucine, or norleucine
  VARIANT 7..8: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  REPEAT 18: note = repeat = 0 or 1
  VARIANT 19..20: note = X=lysine, arginine, or ornithine
  VARIANT 21: note = =tyrosine, repeat=0 or 1
  VARIANT 9: note = X=proline or arginine
XXAAXAXXXAXXXAXXAPXXX

SEQ ID NO: 11 - protein, 21 aa, synthetic construct
  VARIANT 1..2: note = X=lysine, arginine, or ornithine
  VARIANT 19..20: note = X=lysine, arginine, or ornithine
  VARIANT 21: note = X=tyrosine, repeat is 0 or 1
XXAAVALLPAVLLALLAPXXX

SEQ ID NO: 12 - protein, 33 aa, synthetic construct
  VARIANT 1..2: note = X=lysine, arginine, or ornithine
  VARIANT 5: note = X=valine, leucine, isoleucine, or norleucine
  VARIANT 7..9: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  VARIANT 17..18: note = X=lysine, arginine, or ornithine
  VARIANT 19: note = X=tyrosine; repeat is 0 or 1
XXAAXAXXXAXXXAXXAPXXXCVQRKRQKLMPC

SEQ ID NO: 13 - protein, 26 aa, synthetic construct
AAVALLPAVLLALLAPVQRKRQKLMP

SEQ ID NO: 14 - protein, 28 aa, synthetic construct
  DISULFID 17..28: note = cyclized by disulfide bond at C17 and C28
AAVALLPAVLLALLAPCVQRKRQKLMPC

SEQ ID NO: 15 - protein, 14 aa, synthetic construct
  VARIANT 8..9: note = X=leucine, isoleucine, or norleucine
  VARIANT 11..12: note = X=leucine, isoleucine, or norleucine
AAVAPAVXXAXXAP

SEQ ID NO: 16 - protein, 16 aa, synthetic construct
AAVALLPAVLLALLAP

SEQ ID NO: 17 - protein, 10 aa, synthetic construct
  VARIANT 1: note = X=lysine; repeat is 0 or 1
  VARIANT 2: note = X=arginine; repeat is 0 or 1
  VARIANT 10: note = X=lysine; repeat is 0 or 2
XXAAVALLPX

SEQ ID NO: 18 - protein, 12 aa, synthetic construct
  VARIANT 1: note = X=lysine; repeat is 0 or 1
  VARIANT 2: note = X=arginine; repeat is 0 or 1
  VARIANT 12: note = X= lysine; repeat is 0 or 2
XXAVLLALLAPX

SEQ ID NO: 19 - skipped

SEQ ID NO: 20 - protein, 19 aa, synthetic construct
  VARIANT 1: note = X= lysine; repeat is 0 or 1
  VARIANT 2: note = X= arginine; repeat is 0 or 1
  VARIANT 19: note = X= lysine; repeat is 0 or 2
XXAAVALLAAVLLALLAPX

SEQ ID NO: 21 - protein, 16 aa, synthetic construct
TTGTLLPGVLLALVVA

SEQ ID NO: 22 - protein, 19 aa, synthetic construct
  VARIANT 1: note = X=lysine; repeat is 0 or 1
  VARIANT 2: note = X=arginine; repeat is 0 or 1
  VARIANT 19: note = X=lysine; repeat is 0 or 2
XXTTGTLLPGVLLALVVAX

SEQ ID NO: 23 - protein, 16 aa, synthetic construct
TTGTLLPRVLLALVVA

SEQ ID NO: 24 - protein, 19 aa, synthetic construct
  VARIANT 1: note = X=lysine; repeat is 0 or 1
  VARIANT 2: note = X=arginine; repeat is 0 or 1
  VARIANT 19: note = X=lysine; repeat is 0 or 2
XXTTGTLLPGVLLALVVAX

SEQ ID NO: 25 - protein, 10 aa, synthetic construct
  VARIANT 1: note = X=lysine; repeat is 0 or 1
  VARIANT 2: note = X=arginine; repeat is 0 or 1
  VARIANT 10: note = X=lysine; repeat is 0 or 2
XXTTGTLLPX

SEQ ID NO: 26 - protein, 11 aa, synthetic construct
  VARIANT 1: note = X=lysine; repeat is 0 or 1
  VARIANT 2: note = X=arginine; repeat is 0 or 1
  VARIANT 11: note = X=lysine; repeat is 0 or 2
XXVLLALVVAX

SEQ ID NO: 27 - skipped

SEQ ID NO: 28 - protein, 19 aa, synthetic construct
  VARIANT 19: note = X=lysine; repeat is 0 or 1
RAAVALLPAVLLALLAPYX

SEQ ID NO: 29 - protein, 19 aa, synthetic construct
  VARIANT 19: note = X= lysine; repeat is 0 or 1
RAAVALLPAVLLAVLAPYX

SEQ ID NO: 30 - protein, 32 aa, synthetic construct
  VARIANT 1: note = X=lysine; repeat is 0 or 1
  VARIANT 2: note = X=arginine; repeat is 0 or 1
  VARIANT 31: note = X=lysine; repeat is 0 or 1
  REPEAT 32: note = repeat is 0 or 2
XXAAVALLPAVLLALLAPDVRKRQDLEQKMXY

SEQ ID NO: 31 - protein, 19 aa, synthetic construct
  VARIANT 1: note = X= lysine; repeat is 0 or 1
  VARIANT 2: note = X= arginine; repeat is 0 or 1
  VARIANT 19: note = X= lysine; repeat is 0 or 2
XXAAVALLPAVLLALLAPX

SEQ ID NO: 32 - protein, 19 aa, synthetic construct
  VARIANT 1: note = X=lysine; repeat is 0 or 1
  VARIANT 2: note = X=arginine; repeat is 0 or 1
  VARIANT 19: note = X=lysine; repeat is 0 or 2
XXAAVALLAAVLLALLAPX

SEQ ID NO: 33 - protein, 19 aa, synthetic construct
  VARIANT 1: note = X=lysine; repeat is 0 or 1
  VARIANT 2: note = X=arginine; repeat is 0 or 1
  VARIANT 19: note = X=lysine; repeat is 0 or 2
XXTTGTLLPGVLLALVVAX

SEQ ID NO: 34 - protein, 19 aa, synthetic construct
  VARIANT 1: note = X=lysine; repeat is 0 or 1
  VARIANT 2: note = X=arginine; repeat is 0 or 1
  VARIANT 19: note = X=lysine; repeat is 0 or 2
XXTTGTLLPRVLLALVVAX

SEQ ID NO: 35 - protein, 10 aa, synthetic construct
  VARIANT 1: note = X=lysine; repeat is 0 or 1
  VARIANT 2: note = X=arginine; repeat is 0 or 1
  VARIANT 10: note = X=lysine; repeat is 0 or 2
XXTTGTLLPX

SEQ ID NO: 36 - protein, 12 aa, synthetic construct
  VARIANT 1: note = X=lysine; repeat is 0 or 1
  VARIANT 2: note = X=arginine; repeat is 0 or 1
  VARIANT 12: note = X=lysine; repeat is 0 or 2
XXGVLLALVVAX

SEQ ID NO: 37 - protein, 29 aa, synthetic construct
  VARIANT 1: note = X=lysine; repeat is 0 or 1
  VARIANT 2: note = X=arginine; repeat is 0 or 1
XXAAVALLPAVLLALLAPCVQKRQKLMPC

SEQ ID NO: 38 - protein, 20 aa, synthetic construct
KKAAVALLPAVLLALLAPKK

SEQ ID NO: 39 - protein, 20 aa, synthetic construct
RRAAVALLPAVLLALLAPRR

SEQ ID NO: 40 - protein, 20 aa, synthetic construct
RRAAVALLPAVLLALLAPRK

SEQ ID NO: 41 - protein, 21 aa, synthetic construct
RKAAVALLPAVLLALLAPRKY

SEQ ID NO: 42 - protein, 11 aa, synthetic construct
KRAAVALLPKK

SEQ ID NO: 43 - protein, 13 aa, synthetic construct
KRAVLLALLAPKK

SEQ ID NO: 44 - protein, 20 aa, synthetic construct
KRAAVALLAAVLLALLAPKK

SEQ ID NO: 45 - protein, 20 aa, synthetic construct
KRTTGTLLPGVLLALVVAKK

SEQ ID NO: 46 - protein, 20 aa, synthetic construct
KRTTGTLLPGVLLALVVAKK

SEQ ID NO: 47 - protein, 11 aa, synthetic construct
KRTTGTLLPKK

SEQ ID NO: 48 - protein, 12 aa, synthetic construct
KRVLLALVVAKK

SEQ ID NO: 49 - protein, 11 aa, synthetic construct
KRAAVALLPKK

SEQ ID NO: 50 - protein, 16 aa, synthetic construct
AAVALLPAVLLALLAP

SEQ ID NO: 51 - protein, 29 aa, synthetic construct
AAVALLPAVLLALLAPCYVQRKRQKLMPC

SEQ ID NO: 52 - protein, 28 aa, synthetic construct
AAVALLPAVLLAVLAPCVQRKRQKLMPC

SEQ ID NO: 53 - protein, 28 aa, synthetic construct
AAVALLPAVLLALLAPCVQRDEQKLMPC

SEQ ID NO: 54 - protein, 28 aa, synthetic construct
AAVALLPAVLLAVLAPCVQRDEQKLMPC

SEQ ID NO: 55 - protein, 21 aa, synthetic construct
  VARIANT 1..2: note = X=lysine, arginine, or ornithine
  VARIANT 5: note = X=valine, leucine, isoleucine, or norleucine
  VARIANT 7..8: note = X=valine, leucine, isoleucine, or norleucine
  VARIANT 11..13: note = X=valine, leucine, isoleucine, or norleucine
  VARIANT 15..16: note = X=valine, leucine, isoleucine, or norleucine
  REPEAT 18: note = Repeat is 0 or 1
  VARIANT 19..20: note = X=lysine, arginine, or ornithine
  VARIANT 21: note = X=tyrosine; repeat is 0 or 1
XXAAXAXXPAXXXAXXAPXXX